(19) United States
(12) Patent Application Publication
(10) Pub. No.: US 2006/0257493 A1
(43) Pub. Date: Nov. 16, 2006

(54) Nanoparticulate delivery systems for treating multi-drug resistance

(76) Inventors: Mansoor M. Amiji, Attleboro, MA (US); Dinesh B. Shenoy, Boston, MA (US); Lilian van Vlerken, Brookline, MA (US)

Correspondence Address: WEINGARTEN, SCHURGIN, GAGNEBIN & LEBOVICI LLP, TEN POST OFFICE SQUARE, BOSTON, MA, 02109, US

(21) Appl. No.: 11/413,067

(22) Filed: Apr. 27, 2006

Related U.S. Application Data
(60) Provisional application No. 60/675,837, filed on Apr. 28, 2005.

(51) Int. Cl. A61K 31/739; A61K 31/337; A61K 31/138; A61K 9/14

(52) U.S. Cl. 424/489; 514/54; 514/449; 514/651; 977/906

(57) ABSTRACT

An encapsulated delivery system, and, in particular, a nanoparticulate delivery system representing a qualitatively different approach to overcoming multi-drug resistance while simultaneously administering the chosen drug treatment to a patient, e.g., in a site-specific manner, is disclosed. A composition according to the invention includes a therapeutically effective amount of one or more multi-drug resistance reversing agents selected from the group consisting of ceramide and ceramide modulators; and a therapeutically effective amount of a therapeutic agent, wherein the therapeutic agent is different from the one or more multi-drug resistance reversing agents, and the one or more multi-drug resistance reversing agents and the therapeutic agent are encapsulated, preferably co-encapsulated, in a biocompatible, biodegradable delivery vehicle for delivery to a patient in need of treatment, for example, for specific localization at, or higher probability of delivery to, a treatment site in a patient administered the composition. Preferably, the one or more multi-drug resistance reversing agents are ceramide, paclitaxel or tamoxifen.

### CROSS REFERENCE TO RELATED APPLICATIONS

This application claims the priority of U.S. Provisional Application No. 60/675,837, filed Apr. 28, 2005 entitled, NANOPARTICULATE DELIVERY SYSTEMS FOR TREATING MULTIDRUG RESISTANCE, the whole of which is hereby incorporated by reference herein.

### STATEMENT REGARDING FEDERALLY SPONSORED RESEARCH AND DEVELOPMENT

This invention was supported, in whole or in part, by the National Institutes of Health under Grant No. CA-119617. Therefore, the U.S. Government has certain rights in this invention.

### BACKGROUND OF THE INVENTION

A major clinical obstacle in cancer therapy is the development of resistance to a multitude of chemotherapeutic agents, a phenomenon termed as multi-drug resistance (MDR). The development of drug resistance in a small subset of tumor cells is believed to be the cause for tumor survival despite invasive chemotherapy (Harris et al., 1992). Drug resistance can be classified as either inherent or acquired, where inherent drug resistance is caused by genetic predisposition, while acquired drug resistance is developed in response to prolonged antineoplasic treatment. Cancer cells can acquire multi-drug resistance through several molecular mechanisms, and often more than one mechanism may be responsible for the MDR phenotype. Causes for multi-drug resistance include overexpression of membrane spanning ATP-dependant drug efflux pumps from the ABC transporter family, modifications in glutathione-S-transferase activity, alterations in DNA repair mechanisms and modification of apoptotic signaling (Harris et al., (1992)). Currently, MDR in cancer therapy is treated by systemic administration of two or more chemotherapeutic agents that have different mechanisms of action. Unfortunately, many tumor cells, such as breast cancer cells and ovarian cancer cells, acquire resistance to multiple therapeutic agents after such treatment protocols. The systemic toxicity of these chemotherapeutic agents is also a major clinical limitation.

Another cause of acquired drug resistance is the general problem of lack of sufficient concentration and short residence time of therapeutic agents for reaching a target, e.g., a tumor mass, in vivo and, especially, lack of diffusion of the drug into the core interstitial spaces of the tumor mass (Jain, R. K., 2003; Galmarini et al., 2003). Numerous site-specific drug delivery systems have been developed, which have had various levels of success in increasing the therapeutic benefit of an administered drug by delivering a greater amount of the dose at the target site, thus minimizing the amount of the therapeutic agent that accumulates at non-specific targets. As one example, poly(ε-caprolactone) (PCL) nanoparticles have been found to be useful drug delivery carriers for such tumor-targeted delivery (Chawla et al., 2002). The alkyl structure of the polymer efficiently encapsulates hydrophobic compounds and allows for slow degradation of the particle for extended release of the drug. Surface modification of the colloidal carrier with an agent such as a poly(ethylene oxide)-poly(propylene oxide) (PEO-PPO-PEO) triblock copolymer has been found to improve the solubility of the nanoparticle in the aqueous environment of the body, while also repelling plasma proteins, decreasing immune activation, and increasing circulation time of the nanoparticles in the plasma. The nanoparticles then can accumulate preferentially at the tumor site due to the enhanced permeability and retention effects; long circulation times of the drug-containing polymeric nanoparticles increases the probability that the nanoparticles will reach the tumor vasculature where they then easily can extravasate through the fenestrations in tumor capillaries into the tumor mass to deposit the drug at the intended site. By this mechanism, concentrations of drug inside the tumor cell can be 10-100 fold higher than when administering free drug (Kaul et al., 2002). In addition, nanoparticles are internalized in cells by non-specific endocytosis, leading to increased intracellular localization of the drugs at their molecular targets.

However, even with the arsenal of this site-specific targeting ability in the resources of physicians, the problem of MDR is becoming inexorably worse. New methods of approaching the problem of MDR are needed.

### BRIEF SUMMARY OF THE INVENTION

The invention is directed to an encapsulated delivery system, and, in particular, a nanoparticulate delivery system that represents a qualitatively different approach to overcoming MDR while simultaneously administering the chosen drug treatment to a patient, e.g., in a site-specific manner.

Thus, in one aspect, the invention features a therapeutic composition that includes a therapeutically effective amount of one or more multi-drug resistance reversing agents selected from the group consisting of ceramide and ceramide modulators; and a therapeutically effective amount of a therapeutic agent, wherein the therapeutic agent is different from the one or more multi-drug resistance reversing agents, and the one or more multi-drug resistance reversing agents and the therapeutic agent are encapsulated, preferably co-encapsulated, in a biocompatible, biodegradable delivery vehicle for delivery to a patient in need of treatment, for example, for specific localization at, or higher probability of delivery to, a treatment site in a patient administered the composition. Preferably, the one or more multi-drug resistance reversing agents are ceramide, paclitaxel or tamoxifen. Exemplary therapeutic agents include the antibiotics (such as the penicillins, cephalosporins, and aminoglycosides), antiviral agents (such as HIV reverse transcriptase and protease inhibitors), and pro-apoptotic anticancer agents (such as vincristine and campothecin). Preferred delivery vehicles include biodegradable polymeric nanoparticles (Shenoy et al., 2005; Gref et al., 1994) and nanoemulsions, such as are described in U.S. Provisional Application No. 60/740,602, filed Nov. 29, 2005, hereby incorporate by reference herein, and in PCT publication No. WO 2004/000274 (Application No. PCT/HU2003/000049, filed Jun. 18, 2003). Alternative delivery vehicles include micellar nanocarriers.

In another aspect, the invention is directed to a therapeutic composition including a therapeutically effective amount of two or more compounds selected from the group consisting of ceramide and ceramide modulators, wherein the two or more compounds are co-encapsulated in a biocompatible, biodegradable delivery vehicle for specific and simultaneous delivery to a treatment site in a patient administered the composition. Preferably, the two or more compounds are selected from the group consisting of ceramide, paclitaxel and tamoxifen, and most preferably include ceramide and paclitaxel. Exemplary therapeutic agents include all those discussed above.

In a related aspect, the invention is directed to a method of treating cancer in a patient comprising administering to the patient a therapeutically effective amount of one or more of the compositions according to the invention. Preferably, a composition for treating cancer includes the compounds ceramide and one or more of paclitaxel, vincristine or campothecin.

A further aspect of the invention is directed to a method of treating cardiovascular disease in a patient comprising administering to the patient a therapeutically effective amount of one or more of the compositions according to the invention. Preferably, a composition for treating cardiac disease includes the compounds ceramide and paclitaxel and is administered to the patient as part of a coating on a stent.

In another aspect, the invention is directed to a method of treating HIV/AIDS in a patient comprising administering to the patient a therapeutically effective amount of one or more of the compositions according to the invention. Preferably, a composition for treating HIV/AIDS includes the compounds ceramide and saquinavir.

Thus, in general, the method of the invention is directed to treating a patient comprising administering to the patient a therapeutically effective amount of one or more of the compositions according to the invention. The active agents in the composition of the invention can be co-encapsulated or the agents can be encapsulated separately but incorporated into the same composition. Alternatively, the separately encapsulated active agents can be administered simultaneously or sequentially in separate compositions. The preferred delivery route is by enteral (oral) or parenteral administration.

The terms “multi-drug resistance reversing agents” and “multi-drug resistance sensitizers” are understood to refer to compounds that can be used to treat, and, preferably, to overcome, drug resistance in a patient. The resistance of the patient may be to multiple drugs or to an individual drug and still be treated by the methods of the invention.

As used herein, the term “ceramide modulator” is understood to mean a compound that either enhances the endogenous production of ceramide or inhibits the degradation of ceramide. For example, a compound that enhances the production of ceramide can be an agonist of ceramide synthase, such as paclitaxel or rapamycin, and an inhibitor of the degradation of ceramide can be an antagonist of glucosylceramide synthase, such as tamoxifen.

### DESCRIPTION OF THE DRAWINGS

Other features and advantages of the invention will be apparent from the following description of the preferred embodiments thereof and from the claims, taken in conjunction with the accompanying drawings, in which:

FIG. 1 shows, in abbreviate form, the life cycle of ceramide in a cell;

FIGS. 2A and 2B show intracellular accumulation of PEO-PCL nanoparticles loaded with rhodamine-PTX (red) and NBD-CER (green) in SKOV3 cells at 6 hours incubation. The left panel shows the combined fluorescent signal of PTX and CER loaded nanoparticles, while the right panel shows an overlay of fluorescence on the DIC image of the cells;

FIG. 3 shows percent cell viability after 6-day administration of the paclitaxel and C6-ceramide co-treatment compared with paclitaxel treatment alone, delivered as either free drug in solution or encapsulated in PCL-PEO nanoparticles (n=8/group/cell-type); and

FIGS. 4A and 4B show percent apoptosis induction after 24 hours treatment with PTX (10 nM) vs. PTX (10 nM) in combination with CER (10 μM) in (a) SKOV3 and (b) SKOV3TR. Control refers to a negative control (no treatment). (n=6 samples/group/treatment).

### DETAILED DESCRIPTION OF THE INVENTION

Paclitaxel (PTX) is an anti-tumor chemotherapeutic, derived from the bark of the Pacific yew tree (Taxus brevifolia), that is widely used in the treatment of solid tumors, particularly of the breast and ovaries (Khayat et al., 2002). PTX exerts its cytotoxicity by inducing tubulin polymerization resulting in unstable microtubules, which interferes with mitotic spindle function and ultimately arrests cells in the G2/M phase of mitosis (Bhalla, K. N., 2003). Tumor cells exposed to PTX treatment then, as a result, undergo programmed cell death (apoptosis), which is essentially how PTX exerts its antitumor effect. Although it is understood that cell cycle arrest results in activation of the apoptotic signaling cascade, recent studies suggest that PTX therapy may also cause direct accumulation of endogenous ceramide, a lipid with function as a cellular second messenger in apoptosis (Charles et al., 2001).

Ceramide (CER) is derived intracellularly by hydrolysis of the lipid sphingomyelin, but it can also be produced de novo through N-acylation of sphinganine. Accumulation of endogenous CER, produced by either hydrolysis or de novo formation, is known to occur in response to several stimuli, such as growth factor deprivation, pro-inflammatory signals, exposure to increased temperature and radiation, and other stressors such as chemotherapeutics and related cytotoxic agents (Senchenkov et al., 2001). Among such stimuli, paclitaxel has been shown to elevate intracellular CER levels in breast tumor cells (Charles et al., 2001). Accumulation of intracellular CER is implicated in the cellular responses to stress, such as apoptosis and cell cycle arrest, where CER functions as a second messenger in the signaling cascade that initiates these responses. In fact, studies have shown that administration of exogenous CER analogs, particularly C2- and C6-ceramide, encourages cell death by apoptosis and inhibition of tumor growth in several tumor models (Selzner et al., (2001)). While active CER is accrued by sphingomyelin hydrolysis or de novo production from sphinganine in the cell, CER can subsequently be further metabolized by glycosylation to yield glucosylceramide, a non-toxic form of CER that is not implicated in the initiation of apoptosis (Lavie et al., 1996). The enzyme glucosylceramide synthase (GCS), also known as CER glucosyltransferase or UDP-glucose-N-acylsphingosine D-glucosyltransferase, is responsible for this inactivation of CER (Lavie et al., 1996). Several MDR tumor cell lines have exhibited elevated levels of non-cytotoxic glucosylceramide and corresponding elevated levels of GCS (Lavie et al., 1996), and clinical studies have suggested that glucosylceramide levels are elevated in tumor specimens of breast cancer and melanomas that were poorly responsive to chemotherapy (Lucci et al., 1998).

Building on these findings, the inventors have recognized not only the importance of CER in the mediation of the cytotoxic response to anti-tumor chemotherapeutics, but also that inhibiting apoptotic signaling by inactivating endogenous ceramide is an important mechanism whereby tumors develop multi-drug resistance. With this insight, the inventors developed the drug delivery system according to the invention to overcome the barriers of MDR tumor therapy by administering a ceramide modulator to the patient in need of treatment to elevate intracellular CER levels in the patient while simultaneously, in a targeted manner, co-administering the desired therapeutic agent.

The system of the invention was first tested as a treatment to reverse paclitaxel resistance in an ovarian tumor cell line (SKOV3). The ceramide modulator and the therapeutic agent were co-administered in poly(ethylene oxide)-modified poly(ε-caprolactone) (PEO-PCL) nanoparticles to maximize drug delivery to the tumor site and thus enhance the antitumor response. Exogenous C6-CER was co-administered with paclitaxel in solution or in PEO-PCL nanoparticles, and its therapeutic potential was compared to that with similar delivery of paclitaxel or CER alone.

The results described in the experimental section show that a therapeutic strategy that co-administers the therapeutic agent, in combination with exogenous ceramide or a ceramide modulator, in a delivery vehicle such as polymeric nanoparticles can greatly re-sensitize drug resistant cells, such as ovarian tumor cells, to chemotherapy. The results demonstrate the great potential for clinical use of this therapeutic strategy to overcome MDR.

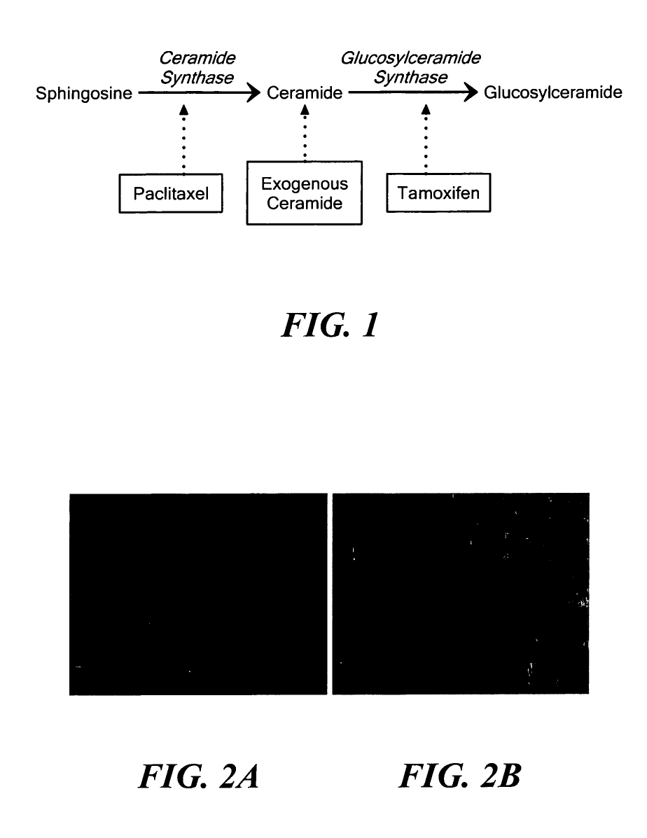

Ceramide is generated from sphingosine by the action of ceramide synthase. The addition of glucose to ceramide yields the non-toxic metabolite glucosylceramide by the action of glycosylceramide synthase. As shown in FIG. 1, the strategy according to the invention for overcoming cellular resistance is to modulate intracellular ceramide concentration by any one or all of the actions of increasing the production of intracellular ceramide by stimulating the activity of ceramide synthase, exogenous administration of ceramide, and inhibiting the metabolism of intracellular ceramide by antagonizing the action of glycosylceramide synthase. Thus, one strategy to increase intracellular ceramide concentrations would involve paclitaxel administration to increases endogenous ceramide production, exogenous ceramide administration, and tamoxifen administration to inhibit ceramide metabolism.

Use

The invention is useful for the treatment of any disease or condition where drug resistance has been encountered, such as cancer, viral and bacterial infections, and cardiovascular diseases. For cancer treatment, co-administration of ceramide or a ceramide modulator in, e.g., a nanoparticulate formulation will enhance therapeutic efficacy with significantly less toxicity. Anti-infective therapy according to the present invention, e.g., administration of an antibiotic or antiviral agent along with ceramide or a ceramide modulator, will enhance the efficacy of the therapeutic agent against bacterial or viral diseases, respectively. In cardiovascular therapy, ceramide or a ceramide modulator co-administration is expected to substantially improve the anti-proliferative effect of paclitaxel, rapamycin, and other agents used in treatment of restenosis.

The therapeutic compositions of the invention may be administered orally, topically, or parenterally (e.g., intranasally, subcutaneously, sublingually, intracranially, intramuscularly, intravenously, or intra-arterially) by routine methods in pharmaceutically acceptable inert carrier substances. For example, the compositions of the invention may be administered in a sustained release formulation using a biodegradable biocompatible polymer, or by on-site delivery using implants, catheters, gels, micelles or liposomes; in other words, the encapsulated active agents may be further encapsulated for sustained release or directed on-site delivery. Other exemplary delivery systems include stents, microchips, implants, patches, ointments, catheters and microspheres (larger polymeric particles).

The compositions of the invention can be administered in a formulation wherein the dosage of exogenous ceramide, or the ceramide modulators paclitaxal and tamoxifen, is, e.g., 1.0 mg/kg/day to 200 mg/kg/day, and preferably 10 mg/kg/day to 100 mg/kg/day. The dosage of the therapeutic agent contained in the composition according to the invention is according to standard protocols known to those of ordinary skill in the art and is, preferably, at a lower dosage due to the benefits of the present invention. Optimal dosage and modes of administration can readily be determined by conventional protocols.

The following examples are presented to illustrate the advantages of the present invention and to assist one of ordinary skill in making and using the same. These examples are not intended in any way otherwise to limit the scope of the disclosure.

### EXAMPLE I

### Co-Encapsulation of Ceramide and Paclitaxel in PEO Modified PCL Nanoparticles

PEO modified PCL nanoparticles were prepared by the controlled solvent displacement method using an acetone-water system, and loaded with 10% w/w paclitaxel (PTX) or 20% w/w C6-ceramide (CER) according to the method of Shenoy et al., 2005. The nanoparticles were formed by dissolving the drug/polymer mixture in acetone, followed by gentle addition of the polymer-drug solution to distilled water under rapid magnetic stirring. Following evaporation of the organic solvent, nanoparticles were collected by centrifugation, washed in distilled water, and lyophilized for storage. Nanoparticle preparations were subsequently subjected to size and zeta-potential measurements using a Brookhaven 90Plus analyzer. For visual nanoparticle tracking, identical batches of PEO-PCL nanoparticles were prepared, but loaded instead with 0.1% w/w rhodamine-PTX or 0.1% w/w NBD-CER.

Controlled solvent displacement produced reproducible PEO-PCL nanoparticles with a mean diameter of 211.6±1.8 nm and surface charge of −31.09±1.53, characteristic of PEO-PCL. Trafficking studies with the rhodamine-PTX and NBD-CER labeled nanoparticles, as shown in FIGS. 2A and 2B, visualized how these nanoparticles are engulfed and trafficked towards the center of the cell to deposit their load. This intracellular drug deposit helps avoid the P-glycoprotein mediated drug efflux characteristic of MDR, which could explain the enhanced cell kill effect that nanoparticle-mediated drug delivery has over delivery of free drugs in solution.

In in-vitro cytotoxicity studies, the wildtype (drug sensitive (DS)) human ovarian cancer cell line SKOV3 was maintained in culture alongside an SKOV3TR subculture that was selected for multi-drug resistance in the presence of increasing concentrations of PTX. For cytotoxicity studies, 96-well plates were prepared with each cell type seeded at 5000 cells/well. The DS and MDR cells were subjected to dose-response studies against PTX, CER and PTX combined with CER, delivered as free drugs in solution or delivered in PEO-PCL nanoparticles. All studies were performed alongside a negative control (cell growth medium), a positive control (polyethyleneimine, PEI), and proper vehicle controls. Since PTX is a cell-cycle dependant chemotherapeutic, all treatments were left to proceed undisturbed for 6 days, allowing ample time for all cells to enter mitosis. Resulting cell death/viability was measured by the MTS (formazan) assay.

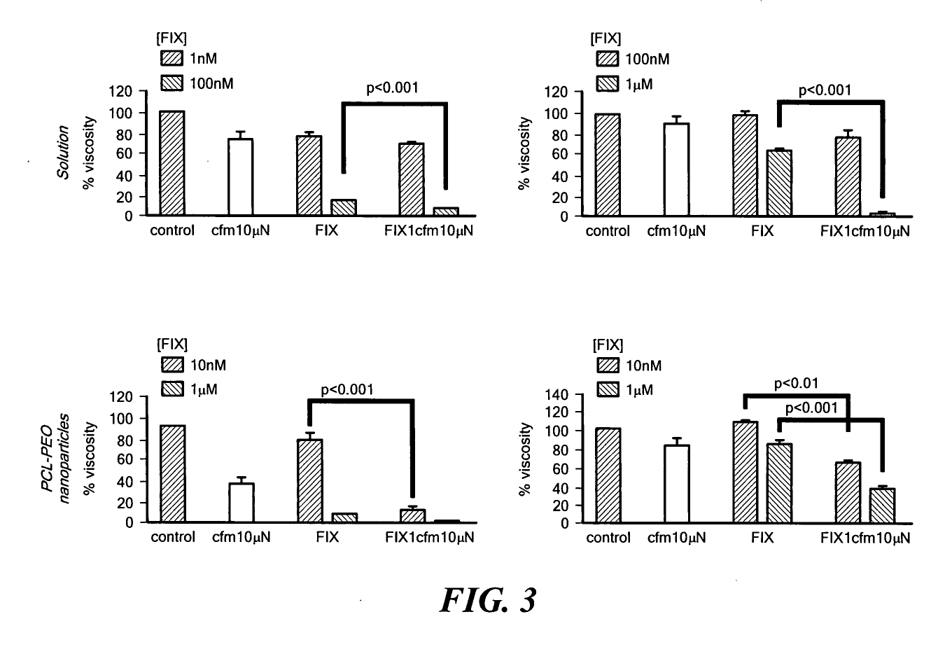

Dose-response studies showed the IC50 of drug sensitive (SKOV3) cells to PTX in solution to be around 5 nM. Therefore, the low-dose (safe-dose) PTX treatment was set at 1 nM while the high-dose (toxic dose) PTX treatment was set at 100 nM. The results shown in FIG. 3 indicate that at 1 nM PTX, 76.03±5.92% of the cells survived, while at 100 nM PTX only 16.37±0.41% of the cells survived, verifying that 100 nM is a highly cytotoxic dose for the wild-type cells. On the other hand, treatment of the SKOV3TR cells with 100 nM PTX resulted in 100.00±5.78% viability, verifying that the MDR cells are indeed significantly resistant to PTX. Treatment with 1 μM of PTX resulted in 65.65±2.16% viability, setting this dose as the experimental cytotoxic dose for the resistant cells. Co-treatment of the SKOV3 cells with 20 μM C6-CER in addition to 1 μM PTX resulted in a significant increase in cell death (2.69±0.51% viability) compared with the PTX treatment alone (p<0.001), while addition of CER did not significantly enhance cytotoxicity at the 100 nM dose of PTX.

Treatment with CER alone at 20 μM did not significantly enhance cytotoxicity either (91.18±7.61% viability). Addition of 10 μM C6-CER to 100 nM PTX dose to the SKOV3 (drug sensitive) cells also resulted in enhanced cell death (7.38±1.25% viability compared with 16.37±0.41% with PTX alone), while, similar to the PTX resistant cells, CER co-treatment did not significantly enhance cytotoxicity at the low PTX dose (70.84±2.62% viability with the co-treatment). CER treatment alone (10 μM dose) did, however, cause slight cytotoxicity in the PTX sensitive cells (75.08±7.86%, p<0.05).

Although the purpose of drug encapsulation within nanoparticles is for the in-vivo benefits of prolonged circulation and tumor-specific drug accumulation, encapsulation of PTX and CER within nanoparticles showed therapeutic benefit over treatment of drug in solution in-vitro as well. Referring again to FIG. 3, treatment with a 10-fold lower concentration of PTX (10 nM) with CER in nanoparticles showed a greater percentage of cell death than treatment with 100 nM PTX with CER (63.98±4.90% viability with nanoparticles vs. 79.78±6.18% viability with treatments in solution). Moreover, encapsulation of the drugs in nanoparticles sensitized the PTX resistant cells to both the high (1 μM) PTX dose as well as the low (10 nM) PTX dose when co-treated with CER (110.58±3.84% viability lowered to 63.98±4.90% viability for the low dose, and 85.09±6.30% viability lowered to 38.15±2.58% viability for the high dose, p<0.001 for both cases). Recall that co-treatment of the drugs in solution resulted only in enhanced cytotoxicity at the high (toxic) dose without affecting cytotoxicity at the low (safe) dose. The same results were obtained with similar treatments in the PTX sensitive cells.

To determine the potential for CER co-administration to result in the hypothesized restoration of apoptotic signaling in the MDR cells, apoptotic activity was measured. A commercially available kit (Vybrant #7, Invitrogen) was used to fluorescently stain apoptotic cells with green fluorescent Yo-Pro and late apoptotic or necrotic cells with propidium iodide in addition to Yo-Pro, distinguishing them from live cells which remained unstained. Blue fluorescent Hoeschst-33342 was used as an internal control for cell count. SKOV3 and SKOV3TR cells were plated in 96-well optical quality plates at a density of 2×10⁴ cells/well. Both cell populations were subjected to treatment with PTX alone, CER alone, PTX in combination with CER, or vehicle (negative control). Treatment proceeded undisturbed for 24 hours to induce apoptosis, at which point cells were stained for apoptotic activity followed by in-situ cytometric analysis of live cells using the icys® microplate cytometry platform (Compucyte Corp., Cambridge, Mass.) that combines laser scanning cytometry with fluorescent microscopy. The iCys® platform allows for simultaneous excitation and absorption of the three dyes for quantitative cell sorting and fluorescent microscopy in one scan. Yo Pro and PI were excited at 488 nm by an argon laser and absorbed at 515-545 nm and 600-635 nm respectively, while Hoechst was excited at 405 nm by a diode laser and absorbed at 445-485 nm. Each sample scan was repeated 4 times, and all treatments were run in triplicate.

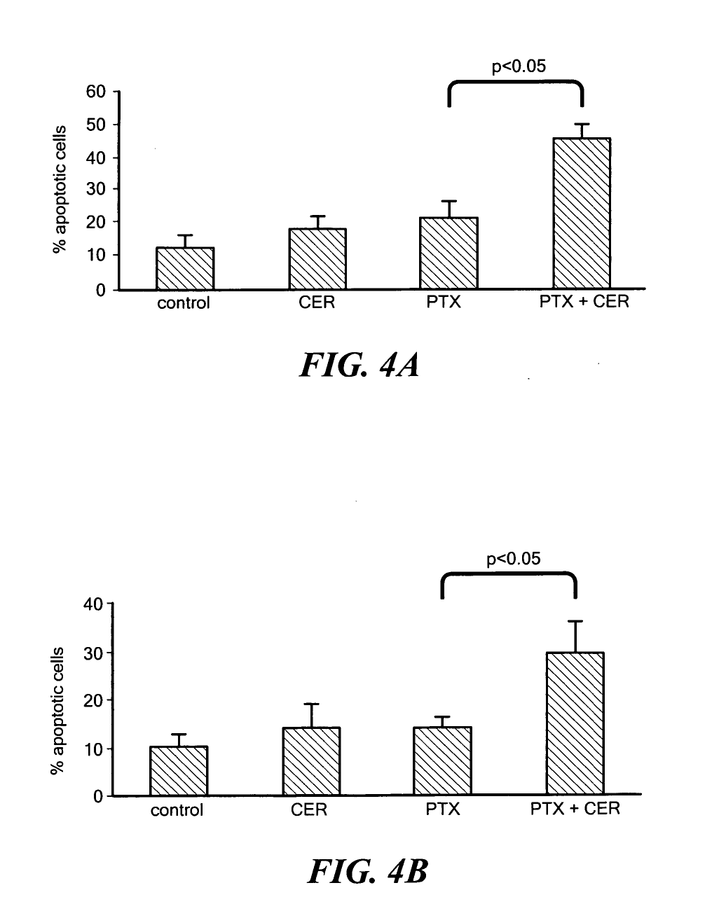

Apoptotic activity was measured 24 hours following treatment initiation by microplate cytometry and confirmed by simultaneous fluorescent microscopy. As shown in FIG. 4B, the co-treatment of PTX/CER results in a twofold increase in the amount of apoptotic activity in the MDR cells over PTX treatment alone, where the concentration of PTX in any of the treatments is merely 10 nM (recall that at 10 nM PTX, 100% of the MDR cells survived). Additionally, as shown in FIG. 4A, exogenous administration of CER with PTX also doubles the amount of apoptotic activity in the drug sensitive SKOV3 cells, although, this result is expected given the decrease in cell viability with PTX/CER co-treatment shown in the cytotoxicity studies.

It is possible, and suggested by the data, that the enhanced apoptotic activity and cell death with the co-treatment in the SKOV3 cells is due to an additive effect of individual PTX and CER cytotoxicities, since there does not appear to be a significant increase in cell death unless the concentration of CER used has a significant cell-kill effect on its own. However, in the MDR cells, there is significant enhancement of cell death when combining concentrations of PTX and CER that individually do not result in significant cell-kill. For example, in the top-right panel of FIG. 3, treatment with CER alone does not produce significant cell death (91.2±7.6% cell viability) while treatment with PTX alone (1 μM) still results in 65.6±2.2% cell viability. However, when combined, treatment at these same concentrations causes nearly 100% cell death (2.7±0.5% cell viability), a phenomenon that, in the MDR cells, is not likely explained by additive PTX and CER cytotoxicities. Thus, it is assumed from the results that a feedback of exogenous CER indeed restores the blocked apoptotic signal initiated by PTX cytotoxicity in the MDR cells, although further studies are needed to confirm this.

### EXAMPLE II

### Preparation and Characterization of Nanoemulsion Formulations

Oil-in-water nanoemulsions useful as delivery vehicles in the compositions of the invention, such as are described in U.S. Provisional Application No. 60/740,602, comprise oil globules having an average size ranging from 5 to 500 nm, with at least one oil, at least one amphiphilic lipid, and an aqueous phase. The nanoemulsions may optionally contain other pharmaceutical aids such as stabilizers, preservatives, buffering agents, antioxidants, polymers and charge inducing agents.

In one embodiment, the nanoemulsions can be formulated by preparing an aqueous phase containing an amphiphilic lipid and homogenizing the solution with lab homogenizer or mixture for 10 min.; preparing an oil phase containing a hydrophobic therapeutic agent and/or a multi-drug resistance reversing agent and mixing the same with a suitable mixing device; heating the solutions of steps 1 and 2 at about 70° C.; and mixing the solutions of step 1 and step 2 together and then homogenizing with a probe sonicator (Sonics and Materials, USA) to obtain nanoemulsions. Alternatively, a high pressure homogenizer (such as a Gauline or an Avestine homogenizer or the like) or a microfludizer can be used for the homogenization step. The number of passes through a high pressure homogenizer/microfludizer can vary depending on the desired particle size of the nanoemulsions.

Alternatively, the oil phase can be mixed with a suitable organic solvent and the organic solvent evaporated to obtain an oil phase film. This film can be then hydrated with the solution of step 1 and the mixture can be homogenized and sized (by a sonicator, high pressure homogenizer or microfludiser) to obtain the nanoemulsions. The hydrophobic therapeutic agent(s) and/or a multi-drug resistance reversing agent(s) can be dissolved in the oil phase or added after formation of the oil film along with the solution of step 1.

To control the particle size distribution of nanoemulsions, they can be filtered using 0.45 micron membrane filters. Nanoemulsions can be lyophillsed in presence of suitable cryoprotectants. Examples of cryoprotectants include but are not limited to glucose, manitol, glycine, high molecular weight polyethylene glycol and other cryoprotectants commonly used in lyophillization of pharmacuticals. The concentration of cryoprotectants can vary from 1% w/v to 80% w/v.

The following nanoemulsion formulations have been prepared:

Example a:

| S. No | Component | | |
|---|---|---|---|
| 1 | Egg Phosphatidylcholine (Lipoid E-80) | 3.00 | gm |
| 2 | Pine nut oil | 25.00 | gm |
| 3 | Paclitaxel* | 8.75 | mL |
| 4 | Purified water | 100 | mL |

*6 mg/mL of marketed injectable formulation (Onxol ™, Ivax Pharmaceuticals Inc, US)

Egg phosphatidylcholine was mixed with water. The mixture was homogenized with a Fisher lab homogenizer (PowerGen 125, Fisher, USA) for 10 min. at half speed. The mixture was heated to 70° C. (Solution A). Pine nut oil and paclitaxel were mixed together, and the solution was heated to 70° C. (Solution B). Solution A was then mixed with solution B, and the mixture was sonicated with a probe sonicator (Sonics and Materials, USA) at 50 percent duty for 20 min. The formed nanoemulsion was measured for particle size by photon correlation spectroscopy (Brookhaven Instruments, USA). The particle size of the emulsion droplets was 195.1±9.8 nm.

Example b:

| S. No | Component | | |
|---|---|---|---|
| 1 | Egg Phosphatidylcholine (Lipoid E-80) | 2.00 | gm |
| 2 | Stearylamine | 1.00 | gm |
| 3 | Sodium Oleate | 0.04 | gm |
| 4 | Glycerol | 2.81 | gm |
| 5 | Pine nut oil | 25.00 | gm |
| 6 | Paclitaxel* | 8.75 | mL |
| 7 | Purified water | 100 | mL |

*6 mg/mL of marketed injectable formulation (Onxol ™, Ivax Pharmaceuticals Inc, US)

Egg phosphatidylcholine, stearylamine, sodium oleate and glycerol were mixed with water. The mixture was homogenized with a Fisher lab homogenizer (PowerGen 125, Fisher, USA) for 10 min. at half speed. The mixture was heated to 70° C. (Solution A). Pine nut oil and paclitaxel were mixed together, and the solution was heated to 70° C. (Solution B). Solution A was then mixed with solution B, and the mixture was sonicated with a probe sonicator (Sonics and Materials, USA) at 50 percent duty for 20 min. The formed nanoemulsion was measured for particle size by photon correlation spectroscopy (Brookhaven Instruments, USA). The particle size of emulsion droplets was 149.0±9.04 nm.

Example c:

| S. No | Component | | |
|---|---|---|---|
| 1 | Egg Phosphatidylcholine (Lipoid E-80) | 2.00 | gm |
| 2 | Deoxycholic acid | 1.00 | gm |
| 3 | Sodium Oleate | 0.04 | gm |
| 4 | Glycerol | 2.81 | gm |
| 5 | Pine nut oil | 25.00 | gm |
| 6 | Paclitaxel* | 8.75 | mL |
| 7 | Purified water | 100 | mL |

*6 mg/mL of marketed injectable formulation (Onxol ™, Ivax Pharmaceuticals Inc, US)

The nanoemulsions of Example c were formulated according to the procedure of Example a, except deoxycholic acid was used as co-surfactant instead of stearylamine. The particle size of emulsion droplets was 147.61±17.04 nm.

Example d:

| S. No | Component | | |
|---|---|---|---|
| 1 | Egg Phosphatidylcholine (Lipoid E-80) | 2.00 | gm |
| 2 | Tween 80 | 1.00 | gm |
| 3 | Sodium Oleate | 0.04 | gm |
| 4 | Glycerol | 2.81 | gm |
| 5 | Pine nut oil | 25.00 | gm |
| 6 | Paclitaxel* | 8.75 | mL |
| 7 | Purified water | 100 | mL |

*6 mg/mL of marketed injectable formulation (Onxol ™, Ivax Pharmaceuticals Inc, US)

The nanoemulsions of Example d were formulated according to the procedure of Example a, except that Tween-80 was used as co-surfactant instead of stearylamine. The particle size of emulsion droplets was 167.61±11.4 nm.

When the nanoemulsions of Example a, b and c were administered to C57BL/6 mice orally at a dose of 25 mg/kg body weight of paclitaxel, an increase in blood AUC (area under concentration curve) was observed when compared with saline diluted paclitaxel solution (marketed injectable solution formulation). This result demonstrates the application of nanoemulsions as a preferred delivery vehicle for compositions according to the invention as they are capable of overcoming the gastrointestinal barrier for systemic delivery of an orally administered drug having an oral bioavailability problem.

### REFERENCES

Bhalla, K. N. (2003) Microtubule-targeted anticancer agents and apoptosis. Oncogene 22:9075-9086.
Charles, A. G., Han, T.-Y., Liu, Y. Y., Hansen, N., Giuliano, A. E., Cabot, M. C. (2001) PTX-induced ceramide generation and apoptosis in human breast cancer cells. Cancer Chemo. Pharm. 47:444-450.
Chawla, J. S., Amiji, M. M. (2002) Biodegradable poly(epsilon-caprolactone) nanoparticles for tumor-targeted delivery of tamoxifen. Int. J. Pharm. 249(1-2):127-138
Galmarini, C. M., and F. C. Galmarini (2003) Multi-drug resistance in cancer therapy: role of the microenvironment. Curr. Opin. Investig. Drugs 4: 1416-1421.
Gref, R., Minamitake, Y., Peracchia, M. T., Trubetskoy, V. S., Torchilin, V. P., and Langer, R., (1194) Biodegradable long circulating polymeric nanospheres. Science, Vol. 263, pp. 1600-1603.
Harris A. L., Hochhauser D. (1992) Mechanisms of multi-drug resistance in cancer treatment. Acta Oncol. 31(2):205-13.
Jain, R. K., Delivery of molecular and cellular medicine to solid tumors. Adv. Drug Del. Revs., 46: 149-168 (2001).
Kaul, G., Amiji, M. (2002) Long-circulating poly(ethylene glycol)-modified gelatin nanoparticles for intracellular delivery. Pharm. Res. 19(7):1061-1067.
Khayat, D., Antoine, E. C., Coeffic, D. (2000) PTX in the management of cancers of the breast and the ovary. Cancer Invest. 18(3):242-260.
Lavie, Y. H.-T. Cao, A. Volner, et al. (1997) Agents that reverse multi-drug resistance, TAM, verapamil, and cyclosporin-A, block glycosphngolipid metabolism by inhibiting ceramide glycosylation in human cancer cells. J. Biol. Chem., 272: 1682-1687.
Lavie, Y., Cao, H., Bursten, S. L., Giuliano, A. E., and Cabot, M. C. (1996) Accumulation of glucosylceramideamides in multi-drug-resistant cancer cells. J. Biol. Chem. 271(32):19530-19536.
Lucci, A., Cho, W. I., Han, T. Y., Giuliano, A. E., Morton, D. L., Cabot, M. C. (1998) Glucosylceramideamide: a marker for multiple-drug resistant cancers. Anticancer Res. 18(1B):475-480.
Radin, N. S., (2003) Killing tumors by ceramide-induced apoptosis: a critique of available drugs. Biochem. J., 371: 243-256.
Shenoy, D. B., Amiji, A. M., (2005)Poly(ethylene oxide)-modified poly (ε-caprolactone) nanoparticles for targeted delivery of tamoxifen in breast cancer. Intl. J. Pharm., Vol. 293, pp 261-270.
Selzner, M., Bielawska, A., Morse, M. A., Rudiger, H. A., Sindram, D., Hannun, Y. A., and Clavien, P.-A. (2001) Induction of apoptotic cell death and prevention of tumor growth by ceramide analogues in metastatic human colon cancer. Cancer Res. 61:1233-1240.
Senchenkov, A., Litvak, D. A., Cabot, M. C. (2001) Targeting ceramide metabolism—a strategy for overcoming drug resistance. J. Nat. Cancer Inst. 93(5):347-357.

While the present invention has been described in conjunction with a preferred embodiment, one of ordinary skill, after reading the foregoing specification, will be able to effect various changes, substitutions of equivalents, and other alterations to the compositions and methods set forth herein. It is therefore intended that the protection granted by Letters Patent hereon be limited only by the definitions contained in the appended claims and equivalents thereof.

What is claimed is:

1. A therapeutic composition comprising
a therapeutically effective amount of one or more multi-drug resistance reversing agents selected from the group consisting of ceramide and ceramide modulators; and
a therapeutically effective amount of a therapeutic agent, wherein said therapeutic agent is different from said one or more multi-drug resistance reversing agents, and
wherein, further, said one or more multi-drug resistance reversing agents and said therapeutic agent are encapsulated in a biocompatible, biodegradable delivery vehicle for delivery to a patient in need of treatment.

2. The therapeutic composition of claim 1, wherein said one or more multi-drug resistance reversing agents and said therapeutic agent are co-encapsulated in said delivery vehicle.

3. The therapeutic composition of claim 1, wherein said one or more multi-drug resistance reversing agents and said therapeutic agent are separately encapsulated in said delivery vehicle.

4. The therapeutic composition of claim 1, wherein said delivery vehicle comprises a targeting agent for specific localization of said one or more multi-drug resistance reversing agents and said therapeutic agent at a target site upon administration of said composition.

5. The therapeutic composition of claim 1, wherein said one or more multi-drug resistance reversing agents is selected from the group consisting of ceramide, paclitaxel, and tamoxifen.

6. The therapeutic composition of claim 1, wherein said therapeutic agent is an antibiotic.

7. The therapeutic composition of claim 1, wherein said therapeutic agent is an antiviral agent.

8. The therapeutic composition of claim 7, wherein said therapeutic agent is saquinavir.

9. The therapeutic composition of claim 1, wherein said therapeutic agent is a pro-apoptotic anticancer agent.

9. The therapeutic composition of claim 1, wherein said therapeutic agent is vincristine or campothecin.

10. The therapeutic composition of claim 1, wherein said delivery vehicle is biodegradable polymeric nanoparticles.

11. The therapeutic composition of claim 1, wherein said delivery vehicle is a nanoemulsion.

12. The therapeutic composition of claim 1, wherein said delivery vehicle is a micellar nanocarriers.

13. A therapeutic composition comprising
a therapeutically effective amount of two or more compounds selected from the group consisting of ceramide and ceramide modulators,
wherein said two or more compounds are encapsulated in a biocompatible, biodegradable delivery vehicle for delivery to a patient in need of treatment.

14. The therapeutic composition of claim 13, wherein said two or more compounds are co-encapsulated in said delivery vehicle.

15. The therapeutic composition of claim 13, wherein said two or more compounds are separately encapsulated in said delivery vehicle.

16. The therapeutic composition of claim 13, wherein said delivery vehicle comprises a targeting agent for specific localization of said one or more multi-drug resistance reversing agents and said therapeutic agent at a target site upon administration of said composition.

17. The therapeutic composition of claim 13, wherein two or more compounds are ceramide and paclitaxel.

18. The therapeutic composition of claim 13, wherein two or more compounds are ceramide and tamoxifen.

19. A method of treating cardiovascular disease in a patient, said method comprising the steps of:
providing a patient in need of treatment for cardiovascular disease; and
administering to said patient a therapeutically effective amount of the composition of claim 1.

20. A method of treating cardiovascular disease in a patient, said method comprising the steps of:
providing a patient in need of treatment for cardiovascular disease; and
administering to said patient a therapeutically effective amount of the composition of claim 13.

21. The method of claim 20, wherein said composition comprises ceramide and paclitaxel.

22. The method of claim 20, wherein, in said administering step, said composition is administered as part of a coating on a stent.

23. A method of treating a disease or condition in a patient in need of treatment, said method comprising the steps of:
providing a patient in need of treatment for said disease or condition; and
administering to said patient a therapeutically effective amount of the composition of claim 1.

24. The method of claim 23, wherein said disease or condition is cancer and wherein said composition comprises ceramide and one or more of paclitaxel, vincristine or campothecin.

25. The method of claim 23, wherein said disease or condition is HIV/AIDS and wherein said composition comprises ceramide and saquinavir.

26. A method of treating a disease or condition in a patient in need of treatment, said method comprising the steps of:
providing a patient in need of treatment for said disease or condition; and
administering to said patient, simultaneously or sequentially, a therapeutically effective amount of one or more multi-drug resistance reversing agents selected from the group consisting of ceramide and ceramide modulators; and
a therapeutically effective amount of a therapeutic agent, wherein said therapeutic agent is different from said one or more multi-drug resistance reversing agents, and
wherein, further, said one or more multi-drug resistance reversing agents and said therapeutic agent are each encapsulated in a biocompatible, biodegradable delivery vehicle.
